(19) United States
(12) Patent Application Publication
(10) Pub. No.: US 2026/0054028 A1
(43) Pub. Date: Feb. 26, 2026

(54) Distributed Feed-forward Psychoacoustic Control

(71) Applicant: Vital Neuro, Inc., New York, NY (US)

(72) Inventors: Kamran Fallahpour, New York, NY (US); John Golden, New York, NY (US)

(21) Appl. No.: 19/105,396

(22) PCT Filed: Aug. 23, 2023
(86) PCT No.: PCT/US2023/030913

§ 371 Date: Feb. 21, 2025

Related U.S. Application Data
(60) Provisional application No. 63/400,135, filed on Aug. 23, 2022.

(51) Int. Cl. A61M 21/00; G10H 1/00; G16H 20/70; G16H 40/67

(52) U.S. Cl. CPC A61M 21/00; G10H 1/0025; G16H 20/70; G16H 40/67; A61M 2021/0027; A61M 2205/33; G10H 2210/111; G10H 2210/125; G10H 2240/131

(57) ABSTRACT

Distributed feedforward-circuitry that uses an environmental information as a feedforward variable includes an environmental-information source and remote circuitry that is remote from the environmental-information source and configured to receive environmental information from the environmental-information source. The remote circuitry includes a machine-learning system, a target-feature set, and a controller. The machine-learning system has been trained to correlate environmental information and psychoacoustic features with mental state. The target feature set, which is generated by the machine-learning system, comprises a psychoacoustic feature for inclusion in a music stimulus that is to be provided to the subject. The controller causes formation of the music stimulus based on the psychoacoustic feature.

### CROSS REFERENCE TO RELATED APPLICATIONS

This application claims priority to U.S. Application No. 63/400,135, filed Aug. 23, 2022, the contents of which are hereby by reference in their entirety.

### BACKGROUND

It is widely accepted that music has an effect on a human's mental state. Thus, it is known to choose music to cause a human to have a particular mood. As an example, certain bugle calls, such as “Taps,” are intended to promote calm whereas other bugle calls, such as “Reveille,” are intended to arouse.

It is also known that individual humans will select music in an effort to achieve particular moods. This require conscious effort on the part of the human. In addition, the palette of music is limited to what is available. One might reasonably expect to select any one of millions of tunes from a streaming service. But even a streaming service cannot provide a musical composition tailored to the listener's unique circumstances.

### SUMMARY

In one aspect, the invention features distributed feedforward-circuitry that uses environmental information as a feedforward variable for providing a music stimulus to be listened to by a subject to urge the subject to achieve a target state of consciousness. Such distributed feedforward-circuitry includes an environmental-information source that provides the environmental information and remote circuitry that is remote from the environmental-information source. The remote circuitry is configured to receive the environmental information from the environmental-information source and includes a machine-learning system, a target-feature set, and a controller. The machine-learning system has been trained to correlate environmental information and psychoacoustic features with mental state. The target feature set, which is generated by the machine-learning system, includes at least one psychoacoustic feature that is to be included in a music stimulus that is to be provided to the subject. The controller is configured to cause formation of the music stimulus so as to includes the psychoacoustic feature.

In some embodiments, the remote circuitry further includes a music-stimulus synthesizer in communication with a music source. In such embodiments, the controller controls the music-stimulus synthesizer based on the target-feature set. Among these are embodiments in which the controller and that assembles, from tracks in a music source, a music stimulus having the psychoacoustic feature. Also among these are embodiments that further include a randomizer that randomly selects a track subset comprising tracks stored in the music source. In these embodiments, each track in the subset has the psychoacoustic feature and the music-stimulus synthesizer uses the randomly-selected track in constructing the music stimulus.

In still other embodiments, the music source includes a music library that includes tracks, among which is a track that includes the psychoacoustic feature.

Some embodiments further include local circuitry that is remote from the remote circuitry, wherein the local circuitry interfaces between the remote circuitry and a headset that provides the music stimulus to the subject. Among these are embodiments that further include a headset. In such embodiments, the local circuitry is in communication with the environmental-information source and with the headset, wherein the local circuitry is configured to provide the environmental variable to the remote circuitry and to provide the headset with the music stimulus received from the remote circuitry.

Still other embodiments include a smartphone that interfaces between the remote circuitry and a headset that provides the music stimulus to the subject. Among these are embodiments that further include a mood controller app that executes on the smartphone.

Embodiments also include a variety of environmental information sources, among which are those that provide information concerning current weather conditions as the feedforward variable, those that provide information concerning past weather conditions as the feedforward variable, those that provide a weather forecast as the feedforward variable, those that provide information concerning ambient lighting as the feedforward variable, and those that provide information concerning ambient lighting as the feedforward variable. Still other embodiments are those in which the environmental-information source includes a sentiment-analysis engine and those in which the environmental-information source includes a news feed or a feed that provides dynamically-changing values of such entities as shares of stock, bonds, tranches of multiple financial instruments, values of commodities, and values of currencies, including cryptocurrencies.

In some embodiments, the environmental-information source is on a warn article, such as a smart watch, smart clothing, or a smart accessory such as a smart belt, a smart ring, smart suspenders, or a smart phone. In still other embodiments, the environmental sensor is a smart appliance that includes internet of things functionality built in, in which case the sensed environmental variable could include the temperature and humidity within a smart refrigerator or smart freezer and the rate of change of temperature in a smart oven or smart air fryer. In still other cases, the environmental sensor is configured to be in a smart fire alarm or a smart carbon monoxide detector in which case the environmental variable is a smoke concentration or a carbon monoxide concentration. In still other embodiments, the environmental-information source is a constituent of the local circuitry that is also connected to the remote circuitry.

Embodiments include a variety of music sources, among which are a music source that includes music that comes from a third-party music engine and that has been pre-categorized by an artificial-intelligence engine and further processed to add psychoacoustic features for operant conditioning and a music source that includes music that has been composed so as to include at least the psychoacoustic feature.

Still other embodiments include those in which the music source is resident on a smartphone that interfaces between the remote circuitry and a headset and those in which the music source is resident on the remote circuitry.

In another aspect, the invention features a method that includes receiving environmental information concerning a subject's environment and using the environmental information as a feedforward variable for providing a music stimulus to the subject. Using the environmental information as a feedforward variable comprises determining that a psychoacoustic feature is expected to cause the subject's mental state to change prior to the subject having detected the environmental information and providing the music stimulus to the subject comprises providing the subject with a music stimulus that includes the psychoacoustic feature.

Some practices of the method further include comprising training a machine-learning system to correlate environmental information and psychoacoustic features with mental state. In such practices, determining that the psychoacoustic feature is expected to cause the subject's mental state to change prior to the subject having detected the environmental information includes using the machine-learning system to generate a target feature set that comprises the psychoacoustic feature.

In some embodiments, the remote circuitry includes application-specific circuitry that includes resistors, capacitors, inductors, transistors, and diodes together with a clock that controls the intervals in which charge is made to move through the various circuit elements. Among the circuit elements are arrays of semiconductor devices that maintain one of two desired states over time and that are made to transition between states at selected times.

The various steps carried out by the remote circuitry have proven to be incapable of being performed in a human mind given its current state of evolution. Indeed, it was for this reason that remote circuitry was required to implement the methods described herein.

Additionally, the various steps carried out by the remote circuitry have proven to be incapable of being performed have also been found to be incapable of being on a generic computer. Thus far, they have only been performed on a non-generic computer.

All attempts to cause the remote circuitry to perform the methods described herein in an abstract manner have thus far failed. Each attempt resulted in performance of the method in a non-abstract manner, where “non-abstract” is defined herein as the converse of “abstract” as that term is used by the Supreme Court of the United States.

The claims are explicitly defined to include only non-abstract implementations of the recited apparatus and methods, where “non-abstract” has been defined as above. Any party who presumes to construe the claims as being abstract in nature would simply be proving that it is possible to improperly construe the claims in a manner inconsistent with express statements to the contrary within the specification.

These and other features will be apparent from the following detailed description and the accompanying figures, in which:

### BRIEF DESCRIPTION OF THE DRAWINGS

FIG. 1 shows an embodiment of circuitry for selecting music having features that are expected to urge a user's brain towards a desired mental state and

FIG. 2 shows a system similar to that in FIG. 1 but with a randomizer that suppresses a user's ability to perceive repeated exposure to the same music feature.

### DETAILED DESCRIPTION

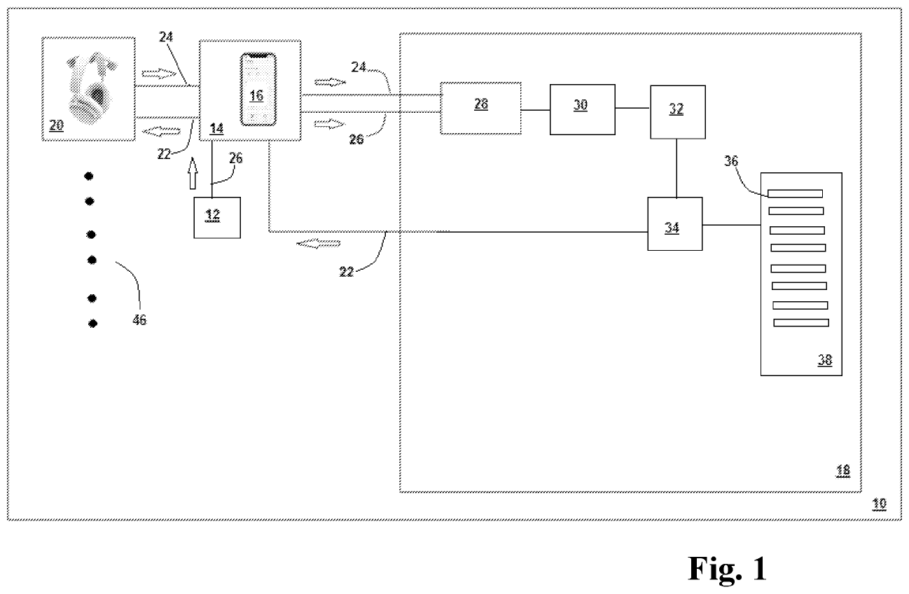

FIG. 1 shows distributed feed-forward circuitry 10 for selecting music that has properties selected to urge a subject's brain towards achieve a desired mental state. The feed-forward circuitry 10 does so based at least in part on an anticipated effect of environmental information, which is used as a feed-forward variable. Examples of mental states towards which the user's brain can be urged include a relaxed state, a stress-reducing state, a state conducive to sleep, a state conducive to enhancement of focus, and a state conducive to greater attention. Other examples include a state of mindfulness, an energetic state, a happy state, and a state of flow.

The distributed feed-forward circuitry 10 relies in part on knowledge of the environmental information and on information concerning how the combination of that environmental information and selected music is likely to affect the subject's mental state. The distributed feed-forward circuitry 10 anticipates an extent to which the environmental information will affect a subject's mental state and uses that information to select an appropriate stimulus to drive the subject towards that mental state or to maintain the subject in that mental state. The knowledge relied upon concerns not just the value of the environmental information but derivatives and integrals thereof over time.

Knowledge concerning environmental information can arise from a variety of sources. For example, knowledge of the subject's location combined with knowledge of current weather conditions is sufficient to make a good prediction of the subject's environment. In those cases in which the subject is in a climate-controlled environment, a sensor within the environment can provide suitable measurements of ambient environmental conditions as well as ambient lighting or sound in the environment. Knowledge of time of day and latitude likewise provides information on lighting experienced by the user.

An example of the use of the derivative would be the use of knowledge indicating, from a weather forecast, that a temperature will soon plummet. Such information may be useful in selecting a stimulus for the subject. An example in which use of an integral is useful is information that a constant drizzle has lasted for several days and is forecast to last several more days. This too may be useful in selecting the stimulus to carry out feedforward control over a subject's mental state.

As used herein, “variable” is not intended to mean a scalar quantity but could include a tuple of scalars that represent different aspects of the environment or a signal indicative of one or more quantities relating to the subject's environment.

In some embodiments, the environmental information is provided as an output of a sentiment-analysis engine based on natural-language analysis, for example of a news feed. In such an embodiment, the sentiment-analysis engine carries out an analysis of incoming news so as to detect unsettling news before the subject has had a chance to see it. The feedforward control system is thus able to use this variable as a basis selecting music to prepare the subject for the unsettling news that is to come.

The environmental knowledge to be used for feedforward control is acquired by an environmental-information source 12. Such an environmental-information source 12 obtains such information and transmits it to local circuitry 14 that executes a mood controller 16. Examples of local circuitry 14 include portable devices, such as a smartphone, a tablet, smart jewelry, a smart watch, and a laptop. Other examples of local circuitry 14 include non-portable devices, such as a personal computer.

The local circuitry 14 acts as an interface between remote circuitry 18 and a headset 20 worn by the subject. The remote circuitry 18 transmits a music stimulus 22 to the headset 20 by way of the local circuitry 14. In addition, the local circuitry 14 receives the subject's selection of a desired state and provides it to the remote circuitry 18.

In operation, the mood controller 16 will have received target instructions 24 from the subject. These target instructions 24 are instructions that are indicative of a target mental state that the subject wishes to achieve. The distributed feed-forward-circuitry 10 also receives sensor information 26 from the environmental-information source 12. This sensor information 26 comprises information about the subject's environment.

The remote circuitry 18 features a machine-learning system 28 that has been trained to correlate environmental states and musical features with mental state. Thus, based on the information that it receives, the machine-learning system 28 is able to provides a target feature-set 30 of psychoacoustic features that music stimulus 22 should have. It then provides this target feature-set 30 to a controller 32.

The controller 32 uses the target feature-set 30 to cause a music-stimulus synthesizer 34 to assemble tracks 36 of music from a music library 38. The music-stimulus synthesizer 34 selects those tracks 36 that include the one or more psychoacoustic features as specified in the target feature-set 30 and causes those tracks 36 to be combined. This results in the music stimulus 22.

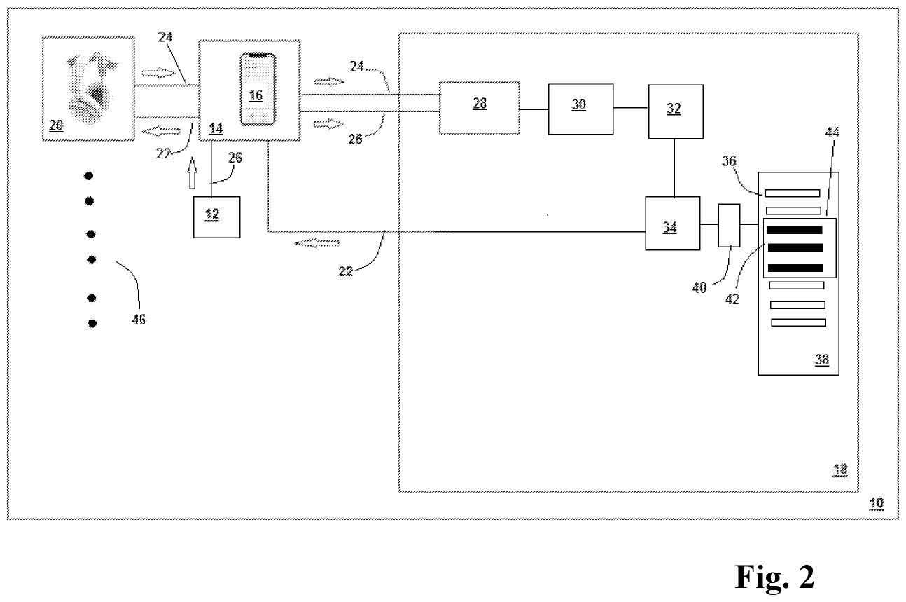

In an alternative embodiment, shown in FIG. 2, a randomizer 40 randomly chooses a track 42 from a subset 44 of tracks 36. Each track 36 in the subset 44 is one that has the desired feature as specified in the target feature set 30. In this embodiment, the subject rarely, if ever, hears the same music twice. Yet, because all tracks 36 in the subset 44 share the same desired feature, the subject continues to receive the desired music stimulus 22. This allows the subject to repeatedly receive the same music stimulus 22 but with almost no likelihood of actually perceiving this repetition.

In both the embodiment of FIG. 1 and FIG. 2, the remote circuitry 18 transmits the music stimulus 22 to the mood controller 16. Upon receipt of the music stimulus 22, the mood controller 16 sends the music stimulus 22 to the headset 20 to be listened to by the subject.

Additionally, in both the embodiment of FIG. 1 and that of FIG. 2, the remote circuitry 18 is available for concurrent use by additional subjects 46 who interact with the remote circuitry 18 in the same manner using corresponding headsets 20 and local circuitry 14. Since the same machine learning system 28 is used for all the subjects 46, it becomes possible for the machine learning system 28 to detect patterns common to all users.

Embodiments further include those in which the music library 38 comprises music that has been composed and psycho-acoustically manipulated to help achieve particular predetermined target feature-sets 30 such as those associated with relaxation, meditation, and focus. Such manipulations include manipulations in tempo, rhythm, instrumentation, melodic patterns, harmonics, instrumentation, frequency emphasis, and orchestration that have been designed to promote a particular state-of-mind corresponding to a target feature-set 30.

Further examples of psychoacoustic manipulation include the manipulation of a track set having one or more tracks. Examples of such manipulation include the filtration of one or more tracks, thus altering their respective spectra, or the addition of binaural beats. Additional examples include causing the listener to listen simultaneously to two tones that are only a few hertz apart such that the two tones interact to produce a third tone that results in psychoacoustic entrainment. In particular embodiments, the third tone has a frequency that depends on the frequency difference between the two tones that are provided. Still other examples include causing the listener to perceive a sound that is not present and doing so by providing the listener with selected input sounds that are selected to be combined by the listener's brain to cause perception of the non-existent sound.

Still other examples of psychoacoustic manipulation include the addition or removal of a particular acoustic effect or controlling the extent of such an acoustic effect. Examples of acoustic effects include reverberation. Another example is that of causing a superposition of phase-delayed copies of a particular signal. Among these are embodiments in which the phase-delayed copies are adjusted in gain, for example by causing copies with larger phase delays to have lower gain. Still other embodiments include those in which psychoacoustic manipulation is carried out by introducing an echo or a delay as well as those that include introducing fuzziness to the sound. Still other embodiments include those in which psychoacoustic manipulation is achieved by adding harmonics to an existing signal. Among these are embodiments in which the harmonics are added to an extent that results in square waves that cause the listener to perceive a fuzzy quality to the music.

Yet other embodiments include those in which psychoacoustic manipulation is carried out by changing the dynamic range of the music, changing its spectral range, or some combination thereof. This would include compression or expansion of either the dynamic or spectral range.

A variety of sources are available for music in the music library 38. In some embodiments, the music is specially composed for the occasion. In others, the music comes from a third-party music engine that has been pre-categorized by an artificial-intelligence engine and further processed to add appropriate conditioning elements as described above. In still other embodiments, the music is resident on the subject's device and therefore need not be streamed at all.

In all such cases, the result is music that has been designed using neuroscientific and psycho-acoustic methods to promote achievement of particular mental states. Such music design includes manipulation of one or more musical and psycho-acoustic variable including tempo, rhythm, tones, including overall frequency balance and/or emphasis on lower or higher frequencies, such as bass and treble frequencies, timbre, musical texture, resonance, entrainment, which promotes a temporal locking of various physiological phenomena, such as motor activity, respiration, heart rate, and brain activity, with an external periodic signal, and overtones, which are used to reinforce perception of a fundamental frequency.

1. An apparatus comprising distributed feedforward-circuitry that uses environmental information as a feedforward variable for providing a music stimulus to be listened to by a subject to urge said subject to achieve a target state of consciousness, said distributed feedforward-circuitry comprising
an environmental-information source that provides said environmental information and
remote circuitry that is remote from said environmental-information source, said remote circuitry being configured to receive said environmental information from said environmental-information source, said remote circuitry comprising
a machine-learning system,
a target-feature set, and
a controller,
wherein said machine-learning system has been trained to correlate environmental information and psychoacoustic features with mental state,
wherein said target feature set, which is generated by said machine-learning system, comprises at least one psychoacoustic feature that is to be included in a music stimulus that is to be provided to said subject, and
wherein said controller is configured to cause formation of said music stimulus so as to includes said psychoacoustic feature.

2. The apparatus of claim 1, wherein said remote circuitry further comprises a music-stimulus synthesizer in communication with a music source and wherein said controller controls said music-stimulus synthesizer based on said target-feature set.

3. The apparatus of claim 1, wherein said remote circuitry further comprises a music-stimulus synthesizer that receives instructions from said controller and that assembles, from tracks in a music source, a music stimulus having said psychoacoustic feature.

4. The apparatus of claim 1, wherein said remote circuitry comprises a music-stimulus synthesizer and a randomizer, wherein the randomizer selects a track randomly from a track subset comprising tracks stored in a music source, wherein each track in said subset has said psychoacoustic feature, and wherein said music-stimulus synthesizer uses said randomly-selected track in constructing said music stimulus.

5. The apparatus of claim 1, wherein said music source comprises a music library that comprises tracks, among which is a track that includes said psychoacoustic feature.

6. The apparatus of claim 1, further comprising local circuitry that is remote from said remote circuitry, wherein said local circuitry interfaces between said remote circuitry and a headset that provides said music stimulus to said subject.

7. The apparatus of claim 1, further comprising local circuitry and a headset, wherein said local circuitry is in communication with said environmental-information source and with said headset, wherein said local circuitry is configured to provide said environmental variable to said remote circuitry and to provide said headset with said music stimulus received from said remote circuitry.

8. The apparatus of claim 1, further comprising a smartphone and a mood controller, wherein said smartphone interfaces between said remote circuitry and a headset that provides said music stimulus to said subject and wherein said mood controller is an app that executes on said smartphone.

9. The apparatus of claim 1, further comprising a smartphone that interfaces between said remote circuitry and a headset that provides said music stimulus to said subject.

10. The apparatus of claim 1, wherein said environmental-information source provides information concerning current weather conditions as said feedforward variable.

11. The apparatus of claim 1, wherein said environmental-information source provides information concerning past weather conditions as said feedforward variable.

12. The apparatus of claim 1, wherein said environmental-information source provides information concerning ambient lighting as said feedforward variable.

13. The apparatus of claim 1, wherein said environmental-information source provides information concerning ambient lighting as said feedforward variable.

14. The apparatus of claim 1, wherein said environmental-information source comprises a sentiment-analysis engine.

15. The apparatus of claim 1, wherein said environmental-information source comprises a news feed.

16. The apparatus of claim 1, wherein said music source comprises music that comes from a third-party music engine and that has been pre-categorized by an artificial-intelligence engine and further processed to add psychoacoustic features for operant conditioning.

17. The apparatus of claim 1, wherein said music source comprises music that has been composed so as to include at least said psychoacoustic feature.

18. The apparatus of claim 1, further comprising a smartphone that interfaces between said remote circuitry and a headset, wherein said music source is resident in said smartphone.

19. A method comprising
receiving environmental information concerning a subject's environment and
using said environmental information as a feedforward variable for providing a music stimulus to said subject,
wherein using said environmental information as a feedforward variable comprises determining that a psychoacoustic feature is expected to cause said subject's mental state to change prior to said subject having detected said environmental information and
wherein providing said music stimulus to said subject comprises providing said subject with a music stimulus that includes said psychoacoustic feature.

20. The method of claim 19,
further comprising training a machine-learning system to correlate environmental information and psychoacoustic features with mental state and
wherein determining that said psychoacoustic feature is expected to cause said subject's mental state to change prior to said subject having detected said environmental information comprises using said machine-learning system to generate a target feature set that comprises said psychoacoustic feature.
